(19) United States
(12) Patent Application Publication
(10) Pub. No.: US 2024/0303600 A1
(43) Pub. Date: Sep. 12, 2024

(54) METHOD AND SYSTEM OF INSURANCE AUTHORIZATION CREATOR AND ANALYSIS FOR APPROVAL TO MITIGATE REJECTION

(71) Applicant: VettedClaims, Inc., San Diego, CA (US)

(72) Inventors: Slim Souissi, San Diego, CA (US); William Tontz, St. Pete Beach, FL (US); Binu Johnson, San Diego, CA (US)

(73) Assignee: VettedClaims, Inc., San Diego, CA (US)

(21) Appl. No.: 18/181,541

(22) Filed: Mar. 9, 2023

(51) Int. Cl. G06Q 10/10; G06Q 40/08; G16H 70/20

(52) U.S. Cl. CPC G06Q 10/10; G06Q 40/08; G16H 70/20

(57) ABSTRACT

A method or system of vetting insurance authorization requests and improving chances of approval can include an application at a server or one or more processors that perform the functions of retrieving and extracting information from an authorization request including a treatment and a diagnosis, identifying treatment guidelines based on the information retrieved and extracted from the authorization request, applying rules that have been converted to a standard format that is human readable to provide a confidence of approval value or indicator, and presenting the confidence value or indicator on a user interface based on the rules applied.

### FIELD

The present disclosure is directed to a method and system for creating insurance authorization forms, and more particularly to a method and system of creating and submitting authorization forms with higher rates of approval.

### BACKGROUND

Guidelines used for insurance approvals are very extensive and complicated (often with multiple conditions/criteria). In many cases, guidelines from more than one authority may have to be used to determine if a treatment would likely be approved-for example, workers compensation approvals in CA are based on guidelines published by CA Medical Treatment Utilization Schedule (MTUS) and Official Disability Guidelines (ODG). Coding all insurance approval guidelines, conditions and criteria from all approval authorities in software is prohibitively expensive. In addition, whenever guidelines are updated/revised by authoritative sources, software needs to be updated, tested and deployed again resulting in high operational cost.

### SUMMARY

In some embodiments, a system for vetting insurance authorization requests and improving chances of approval can include one or more processors, memory coupled to the one or more processors where the computer instructions which when executed by the one or more processors, causes the one or more processors to perform several functions including retrieving and extracting information from an authorization request including a treatment and a diagnosis, identifying treatment guidelines based on the information retrieved and extracted from the authorization request, using rules that have been converted to a standard format that is human readable and separately maintain the rules from each authority issuing the rules, and applying rules to provide a confidence of approval value or indicator. The system can further present the confidence value or indicator on a user interface based on the rules applied and also present a submission button for generating a request for authorization form.

In some embodiments, the one or more processors are further configured to index the treatment and the diagnosis from the authorization request.

In some embodiments, the one or more processors are further configured to fetch a rule file or rule files based on the treatment in the authorization request.

In some embodiments, the one or more processors are further configured to determine if an approval is unconditional or conditional. In some embodiments, they are configured to indicate the approval if the approval is unconditional or all conditional requirements are met. In some embodiments, they are configured to solicit for more information to determine claim status if the approval is conditional.

In some embodiments, the one or more processors are further configured to use configuration files to determine which rule files apply.

In some embodiments, the one or more processors are configured to determine if the rule file is a latest version.

In some embodiments, the one or more processors are configured to present an excerpt of the rule file applied to allow determination of why a claim or authorization is approved or denied.

In some embodiments, presenting the confidence value or indicator on the user interface based on the rules applied includes providing an estimated percentage of chance that a claim will be approved.

In some embodiments, the one or more processors are configured to track claims and feedback to provide an improved prediction analysis.

In some embodiments, the one or more processors are configured to iteratively determine and present the confidence value or indicator on the user interface based on the rules applied as the claim is presented and modified.

In some embodiments, the one or more processors are configured to provide a submission score for every authorization request submitted where the score is dependent on a probability that the authorization request adheres to the rules or insurance guidelines.

In some embodiments, a system for vetting insurance authorization requests and improving chances of approval includes a server and one or more applications executing on the server. The one or more applications can be configured to retrieve and extract information from an authorization request including a treatment and a diagnosis, identify treatment guidelines based on the information retrieved and extracted from the authorization request, use rules that have been converted to a standard format that is human readable, apply the rules to provide a confidence of approval value or indicator and present the confidence value or indicator on a user interface based on the rules applied.

In some embodiments, the authorization request is created with information from a first questionnaire from a patient that includes primarily patient demographic information from the patient. In some embodiments, the authorization request is further created with physician examination notes including CPT codes and/or IDC10 codes. In some embodiments, the authorization request is further created with uploaded files and lab tests. In some embodiments, a second questionnaire is generated and sent to the patient based on the CPT codes entered by the physician.

In some embodiments, the system provides a submission score for every authorization request submitted where the score is dependent on a probability that the authorization request adheres to the rules or insurance guidelines where a red indicator indicates a high probability of rejection of the authorization request and a green indicator indicates a high probability of allowance of the authorization request.

In some embodiments, a method of vetting insurance authorization requests and improving chances of approval comprising an application at a server having one or more applications executing on the server which perform the functions of retrieving and extracting information from an authorization request including a treatment and a diagnosis, identifying treatment guidelines based on the information retrieved and extracted from the authorization request, applying rules that have been converted to a standard format that is human readable to provide a confidence of approval value or indicator, and presenting the confidence value or indicator on a user interface based on the rules applied.

### BRIEF DESCRIPTION OF DRAWINGS

FIG. 1 illustrates an insurance claim submission and analysis system in accordance with the embodiments;

FIG. 2 illustrates another insurance claim submission and analysis system in accordance with the embodiments;

FIG. 3 illustrates a user interface on the portable computer or tablet that illustrates a listing of requests for authorization forms in accordance with the embodiments;

FIG. 4 further illustrates the user interface of FIG. 3 with the ability to create a new entry in accordance with the embodiments;

FIG. 5 further illustrates the user interface of FIG. 3 for a particular patient illustrating a treatment request and an indicator of confidence of approval for each treatment in accordance with the embodiments;

FIG. 6 illustrates a user interface similar to FIG. 5 further including a pop-up entry form for generating a new request, a resubmission, or expedited review in accordance with the embodiments;

FIG. 7 illustrates a user interface similar to FIG. 5 further including a treatment request and a pull-down menu for further detailed body system being treated in accordance with the embodiments;

FIG. 8 illustrates a user interface similar to FIG. 5 further including a treatment request and a pull-down menu for further detailed services or goods required for a particular body system being treated in accordance with the embodiments;

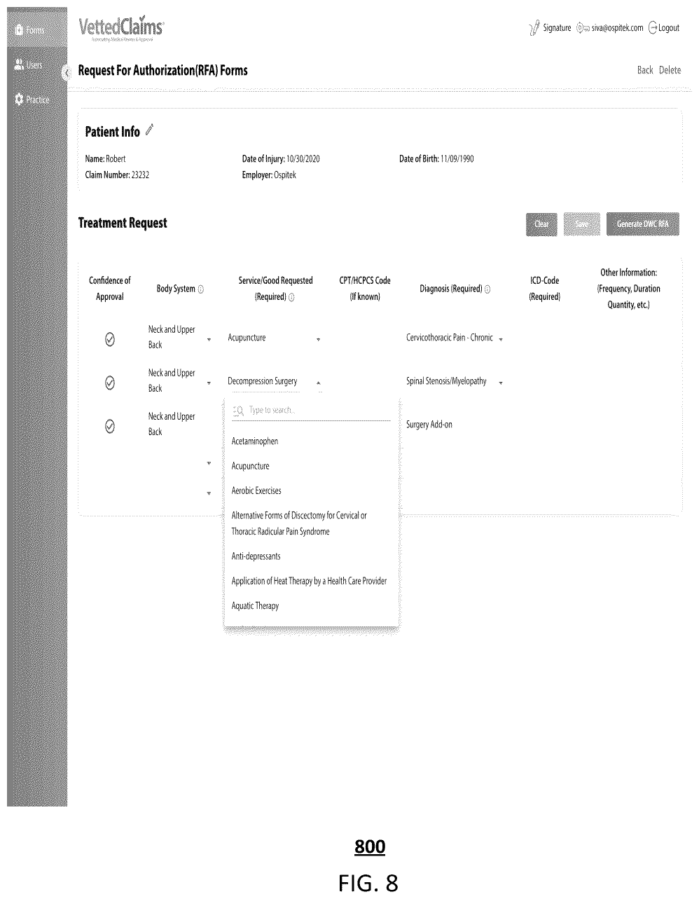

FIG. 9 illustrates a user interface similar to FIG. 5 further including a treatment request and a pull-down menu for further detailed diagnosis in accordance with the embodiments;

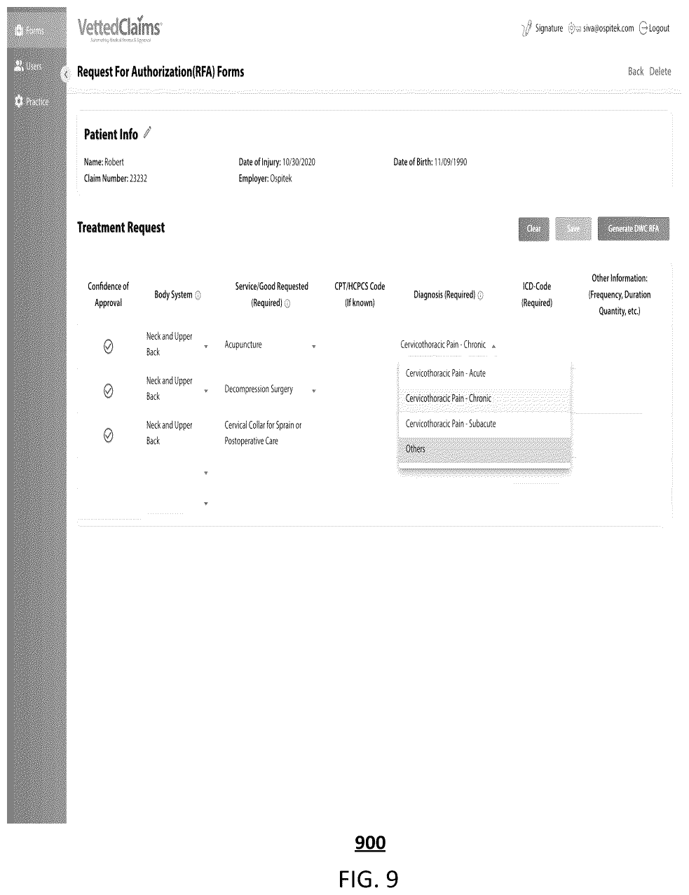

FIG. 10 illustrates another user interface for a particular patient illustrating a treatment request and an indicator of confidence of approval for each treatment proposed in accordance with the embodiments;

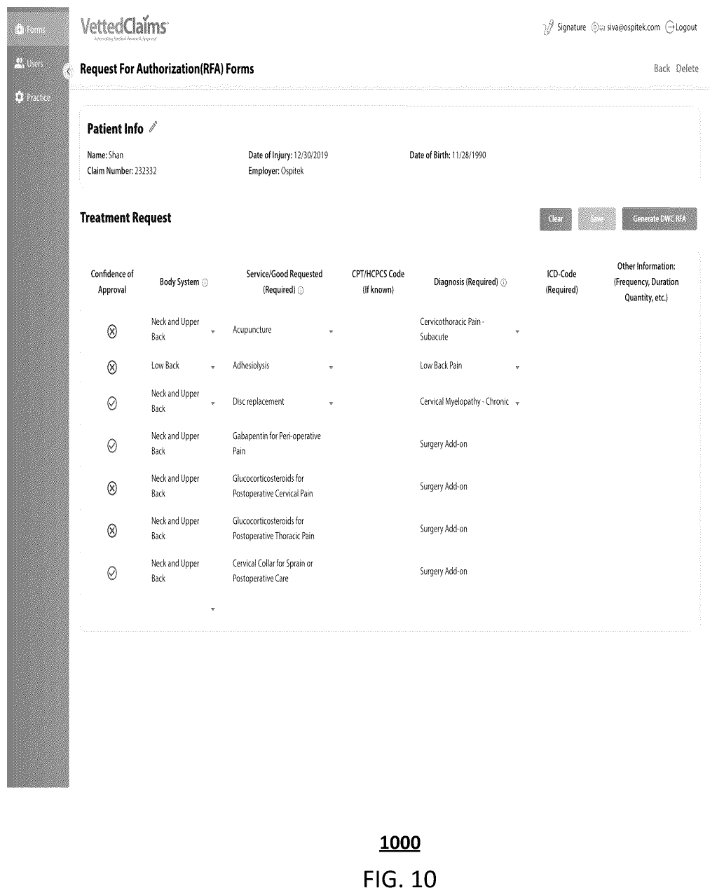

FIG. 11 illustrates a method of generating, analyzing, and submitting a request for approval of insurance having a higher approval rate in accordance with the embodiments; and

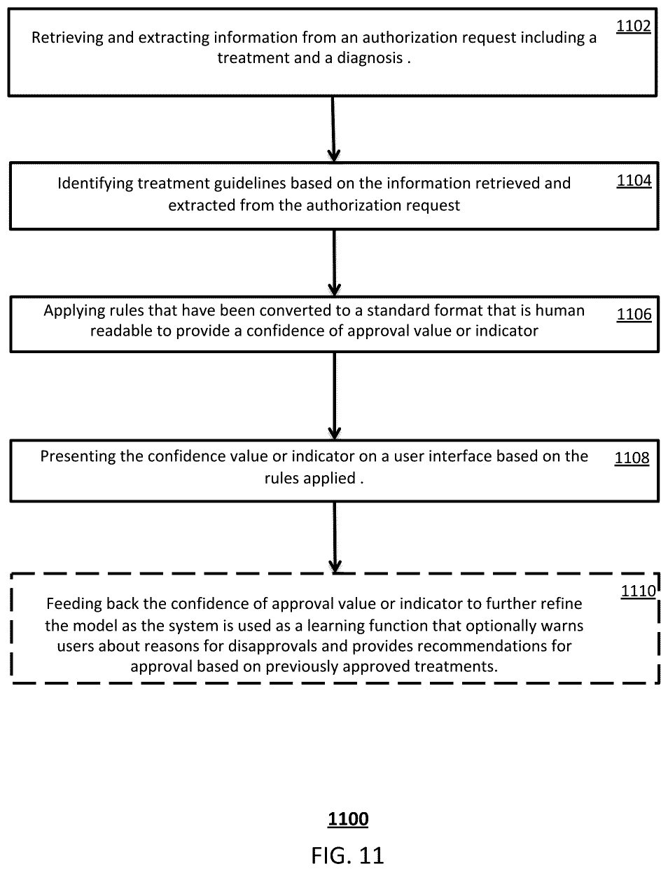

FIG. 12 illustrates a system that can implement the method and user interfaces of FIGS. 1-11 in accordance with the embodiments.

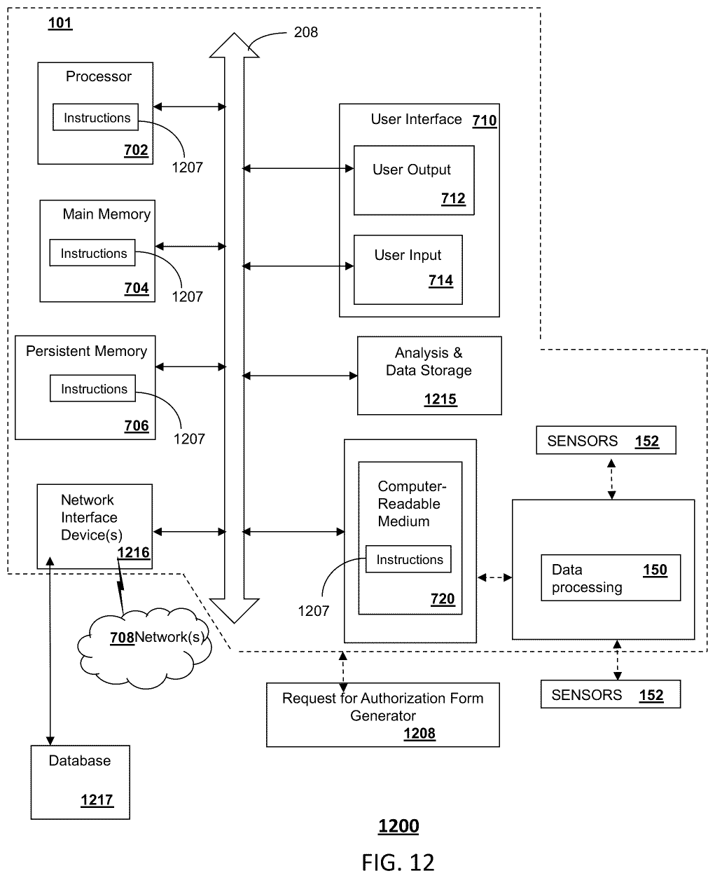

### DETAILED DESCRIPTION

The claimed embodiments can include a web application for a physician's office to create authorization requests for insurance approval for treatments or procedures. While creating an authorization request, the application can automatically evaluate if a proposed treatment is likely to be approved or denied based on approval guidelines published by governing authorities. The embodiments can be adapted for various medical insurance programs run federally or by the various states and can be further tailored for Medicare, Medicaid, Workman's Compensation or other insurance programs

The application in accordance with the embodiments can help physician's offices to reduce insurance denials and approval delays, and further improve their reputation with insurance companies because of high quality authorization requests and high approval rates. The embodiments also help insurance companies by reducing utilization reviews and cost of processing insurance claims. Some of the examples are only illustrative. More particularly, some examples shown illustrate one type of insurance claim for worker's compensation and in the state of California. Obviously, the claimed embodiments are not limited to such context and environment and can easily be adapted for other environment or context that can involve other types of insurance or other states. The scope and intent of the embodiments is to be flexible to cover more states and more types of insurance claims beyond worker's compensation.

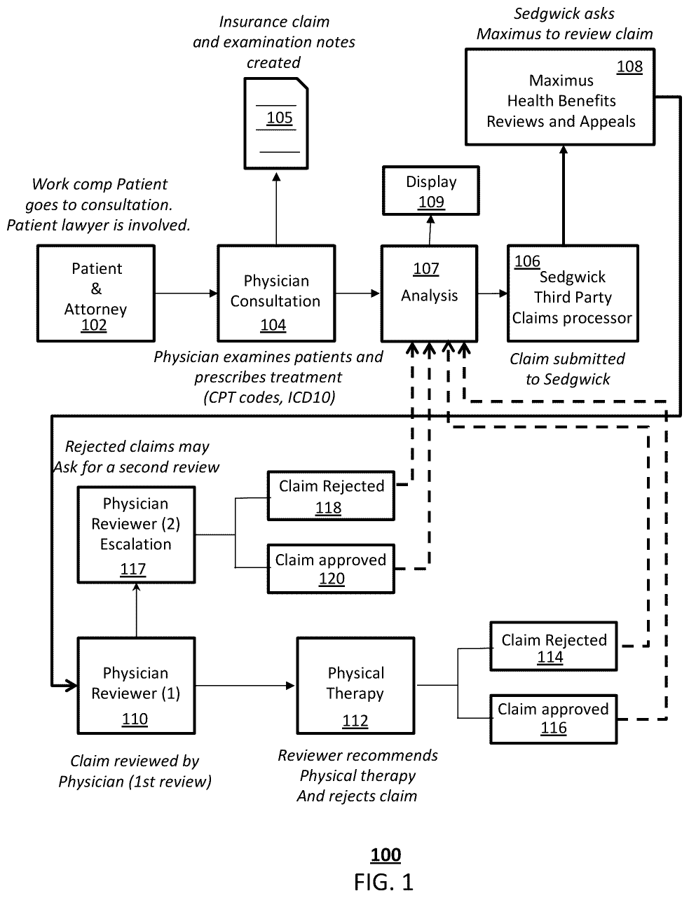

Referring to FIG. 1, a platform or system 100 illustrates how physicians can submit insurance claims that adhere to insurance guidelines that minimize the probability of a claim being rejected. Much of the process can be automated to reduce errors and duplication.

In the case of a workman's compensation patient, such a patient can consult with an attorney at 102 and have a physician examine a patient to prescribe certain treatments at 104. Such treatments can have associated CPT or ICD-10 codes.

Current Procedural Terminology (CPT®) codes offer doctors and health care professionals a uniform language for coding medical services and procedures to streamline reporting, increase accuracy and efficiency. ICD-10 refers to the tenth edition of the International Classification of Diseases, which is a medical coding system chiefly designed by the World Health Organization (WHO) to catalog health conditions by categories of similar diseases under which more specific conditions are listed, thus mapping nuanced diseases to broader morbidities.

As a result of the physician consultation at 104, the physician can create an insurance claim or authorization request along with examination notes at 105. In the California state system, a third party claims processor such as Sedgwick processes workman's compensation claims and other medical claims. Thus, the third party claims processor receives the claim at 106 from the physician. The California system further has a review process for health benefits by an entity call Maximus which is performed at 108. The review process results in a first review by a physician at 110. In some instances, the claim may result in a recommended for physical therapy at 112 where the claim is thereafter either rejected at 114 or approved at 116. In some instances when a claim is rejected, a request for a second review by a physician or escalation is done at 117 where the claim is thereafter either rejected at 118 or approved at 120 after the escalation.

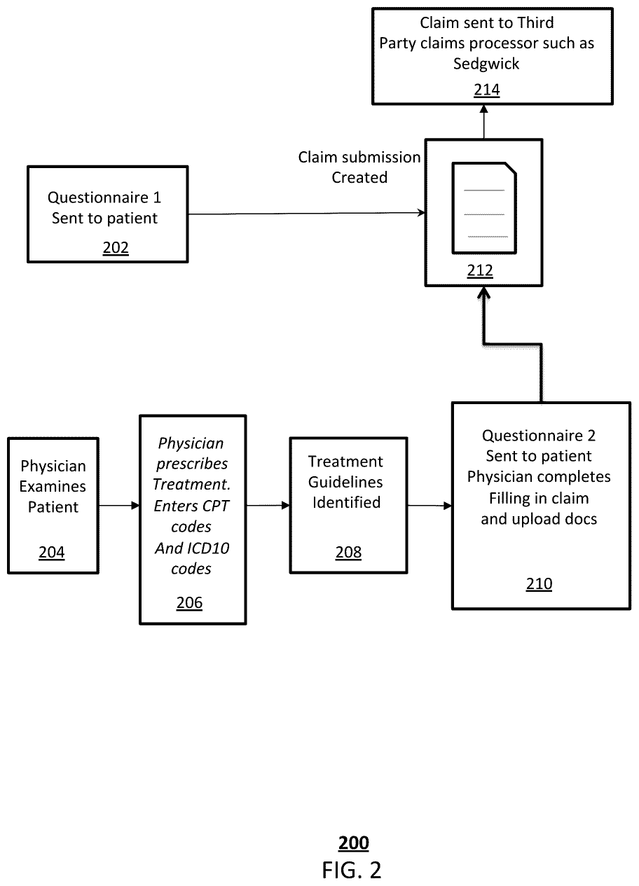

Referring to FIG. 2, a more detailed view of how an insurance claim or authorization request can be created and submitted in a system 200. Initially, a first questionnaire 202 is sent to a patient that contributes to the claim or request being submitted at 212. At 204, the physician examines the patient and prescribes certain treatment or treatments at 206. The treatments can have corresponding CPT and/or ICD-10 codes as noted above which the physician can enter. The system 200 can further identify treatment guidelines at 208. A second questionnaire can be sent to the patient at 210 as a result of the physician entering the aforementioned codes, uploading pertinent files such as X-rays, lab test results and other pertinent information if available or required. The second questionnaire can be created based on the insurance guidelines relevant to the CPT codes (or other codes) enter by the physician. The first and second questionnaires are then used to create the claim submission that can be submitted to the third party processor at 214.

In an overview to the approach or solution in the embodiments, rules that are used for insurance approval decisions are saved in a standardized format that software can read and interpret. Hence software engineers do not write the rules as code, but instead domain experts read official guideline documents and convert them to our standardized format. This enables the system to add or revise insurance approval rules and support new diagnosis or treatments without making any software change.

The standardized format used in the embodiments for defining rules used by insurance approval decisions are indexed by treatment request and diagnosis so that software can efficiently find rules that govern approval for a requested treatment for a given diagnosis.

In the embodiments, policy files are partitioned by treatment request, resulting in small policy files and hence low latency for API calls. When a physician inputs a treatment request and diagnosis, the web application can fetch (the small) policy file and respond to the physician without any perceivable delay.

The format allows specifying unconditional approvals or denials and conditional approvals. In case of conditional approvals, the format allows gathering information from the requesting physician and expressing approval decisions as a decision tree/flow chart.

In some embodiments, insurance approval guidelines from various authorities or sources are maintained separately. A configuration file determines which rules or guidelines will be used by software for the nature of insurance claims (for e.g., if it is a California workers comp case, software would refer to CA MTUS guidelines and use ODG guidelines if the requested treatment is not covered in CA MTUS).

Policy files can hold relevant excerpts from guideline documents so the web application can show the user why a request would be approved or declined. Policy files can also hold references to relevant sections for customers (physician) to learn more.

Policy files hold a reference to the original version of the guidelines doc that was used to create the policy file. An automated periodic job/script can scan all policy files and the latest dates/revision of official files published by authoritative sources to detect if a policy file needs updating.

Policy files are version controlled with the ability to find changes between versions. Policy files also keep track of the author or latest modifier. Versioning allows the embodiments to revert to the previous stable version if a policy file was incorrectly changed and maintaining owner/author information allows the embodiments to audit changes by author.

Policy files can be cataloged based on body systems so it is easy to setup physician accounts based on specialty (e.g. Orthopedics, Ophthalmology, Oncology, etc.).

In some embodiments, policy files are saved in JSON format with keys defined for our purpose. The embodiments certainly contemplate other formats being used other than JSON and still being within the scope of the claimed embodiments.

It is prohibitively expensive to code all rules and guidelines from all sources to make an insurance determination (and to keep the software up to date with all guideline revisions), particularly in an automated fashion. The embodiments herein were made viable because of the techniques disclosed and claimed herein.

The automation in some of the embodiments lies into the capability to train the system to learn from previously submitted claims and based on the approval/disapproval outcome of these claims, the system can automatically estimate the probability of rejection. The system can also look into the history of the claims and find out similarities between claims and decide whether or not they would be accepted. So some sort of a feedback loop into the system that feeds a learning function that warns the users about reasons for disapproval and make recommendations for approvals based on previously approved applications are contemplated.

A further consideration in the embodiments are the various stakeholders that can take advantage of the efficiencies in this new technology including administrators, physicians insurance companies, physician reviewers, and patients. The primary user would be a physician who would likely need to have a login to create patient files, send questionnaires, create claims and manage such claims. Insurance companies would also have a login to a portal to see the submissions by the physicians (or their staff or other agents). The insurance user interface should be able to sort by physician or patient. A physician review should also be able to receive a file to review and approve or disapprove and further publish a decision. Patients can also have certain access, but the platform would need to be HIPPA compliant. In some embodiments, the patients can receive text messages to fill out forms and answer questionnaires as illustrated in FIG. 2.

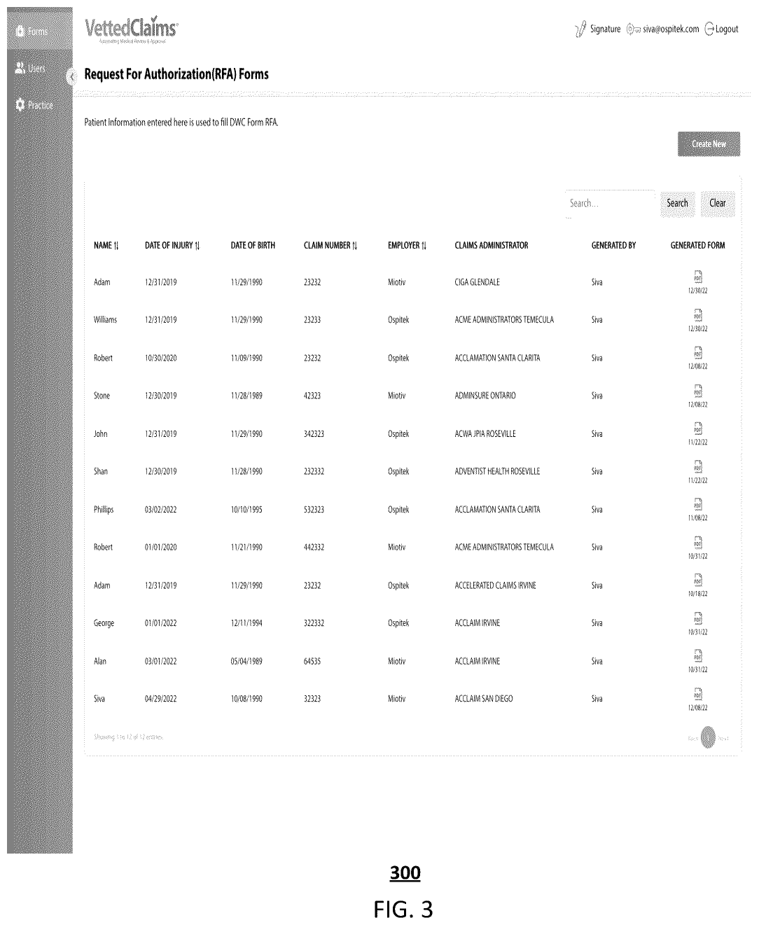

Referring to a user interface 300 of FIG. 3,

Various embodiments of the present disclosure can be implemented on an information processing system. The information processing system is capable of implementing and/or performing any of the functionality set forth above. Any suitably configured processing system can be used as the information processing system in embodiments of the present disclosure. The information processing system is operational with numerous other general purpose or special purpose computing system environments, networks, or configurations.

Examples of well-known computing systems, environments, and/or configurations that may be suitable for use with the information processing system include, but are not limited to, personal computer systems, server computer systems, thin clients, hand-held or laptop devices, notebook computing devices, multiprocessor systems, mobile devices, microprocessor-based systems, set top boxes, programmable consumer electronics, network PCs, minicomputer systems, mainframe computer systems, Internet-enabled television, and distributed cloud computing environments that include any of the above systems or devices, and the like. As noted previously, the data processing can be any number of data processing techniques suited for the generating of electronic charts as contemplated herein.

For example, a user with a mobile device may be in communication with a server configured to implement the system using the aforementioned elements, according to an embodiment of the present disclosure. The mobile device can be, for example, a multi-modal wireless communication device, such as a “smart” phone, configured to store and execute mobile device applications (“apps”). Such a wireless communication device communicates with a wireless voice or data network using suitable wireless communications protocols assuming the networks have the appropriate bandwidth to present data or real time images. Alternatively, the display system can be a computing and monitoring system with or without wireless communications as the case may be. In some embodiments, the device or system can include a server and a tablet or laptop or other mobile computing device.

The system may include, inter alia, various hardware components such as processing circuitry executing modules that may be described in the general context of computer system-executable instructions, such as program modules, being executed by the system. Generally, program modules can include routines, programs, objects, components, logic, data structures, and so on that perform particular tasks or implement particular abstract data types. The modules may be practiced in various computing environments such as conventional and distributed cloud computing environments where tasks are performed by remote processing devices that are linked through a communications network. In a distributed cloud computing environment, program modules may be located in both local and remote computer system storage media including memory storage devices. Program modules generally carry out the functions and/or methodologies of embodiments of the present disclosure, as described above.

In some embodiments, a system includes at least one memory and at least one or more processor of a computer system communicatively coupled to the at least one memory. The at least one processor can be configured to perform a method including methods described above.

According to yet another embodiment of the present disclosure, a computer readable storage medium comprises computer instructions which, responsive to being executed by one or more processors, cause the one or more processors to perform operations as described in the methods or systems above or elsewhere herein.

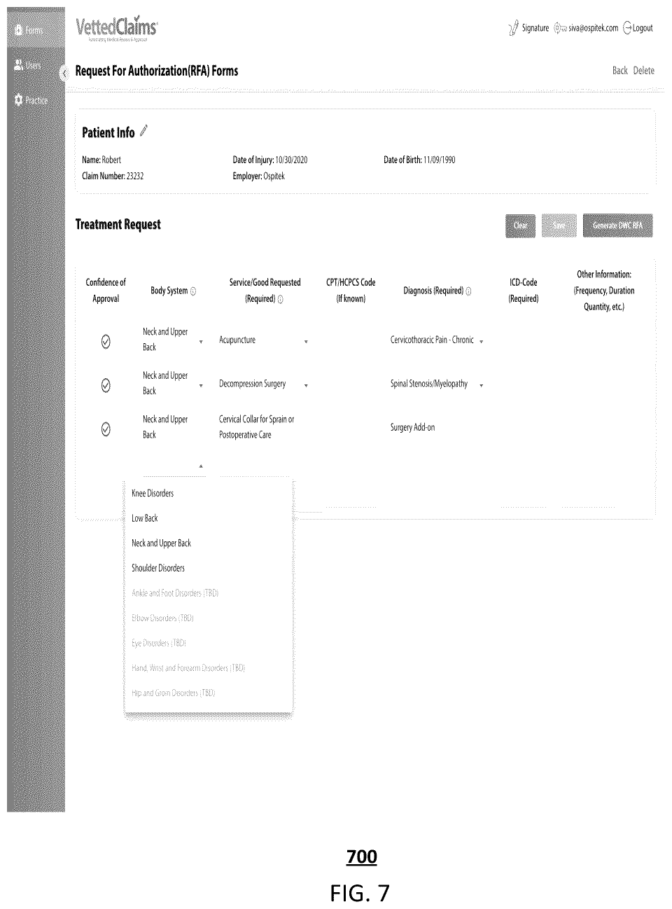

As shown in FIG. 7, an information processing system 101 of a system 700 can be communicatively coupled with the data processing module 150 and a group of client or other devices, or coupled to a presentation device for display at any location at a terminal or server location. According to this example, at least one processor 702, responsive to executing instructions 107, performs operations to communicate with the processing module 150 via a bus architecture 208, as shown. The at least one processor 702 is communicatively coupled with main memory 704, persistent memory 706, and a computer readable medium 720. The processor 702 is communicatively coupled with an Analysis & Data Storage 115 that, according to various implementations, can maintain stored information used by, for example, the data processing module 150 and more generally used by the information processing system 700. The data processing module 150 can be coupled to one or more sensors 152 as needed. Such sensors can be, temperature sensors, oxygenation sensors, heart rate monitors, pressure sensors, weight sensor or scales, location sensors, orientation sensors, rotation sensors, motion sensors, barcode scanners, fingerprint readers, proximity sensors, microphones, cameras, video cameras, motion detectors, thermostats, biometric reading devices (e.g., iris scanners, facial recognition scanners, voice detection devices) and other devices as contemplated herein. Some sensors 152 can be part of the processor 101 itself or operatively coupled to the sensors 152 separate from the processor 101. Optionally, this stored information can be received from the client or other devices. For example, this stored information can be received periodically from the client devices and updated or processed over time in the Analysis & Data Storage 115. Additionally, according to another example, a history log can be maintained or stored in the Analysis & Data Storage 115 of the information processed over time. The data processing module 150, and the information processing system 700, can use the information from the history log such as in the analysis process and in making decisions related to generating forms or fields within forms according to a patient tracking system and patient status or according to a database of best practices for a particular procedure or procedures.

The computer readable medium 720, according to the present example, can be communicatively coupled with a reader/writer device (not shown) that is communicatively coupled via the bus architecture 208 with the at least one processor 702. The instructions 107, which can include instructions, configuration parameters, and data, may be stored in the computer readable medium 720, the main memory 704, the persistent memory 706, and in the processor's internal memory such as cache memory and registers, as shown.

The information processing system 700 includes a user interface (or interfaces) 710 that comprises a user output interface 712 and user input interface 714. Examples of elements of the user output interface 712 can include a display, a speaker, one or more indicator lights, one or more transducers that generate audible indicators, and a haptic signal generator or any of the interfaces illustrated or discussed with respect to the figures or elsewhere in the application. Examples of elements of the user input interface 714 can include a keyboard, a keypad, a mouse, a track pad, a touch screen, a touch pad, a microphone that receives audio signals, a camera, a video camera, a CT-Scanner, or any other scanner that scans images. Some user inputs can be sensors or vice-versa. The received audio signals or scanned images, for example, can be converted to electronic digital representations and stored in memory, and optionally can be used with corresponding voice or image recognition software executed by the processor 702 to receive user input data and commands, or to receive test data for example.

A network interface device 116 is communicatively coupled with the at least one processor 702 and provides a communication interface for the information processing system 101 and any linked IoT Powered e-charting system 108 to communicate via one or more networks 708. The networks 708 can include wired and wireless networks, and can be any of local area networks, wide area networks, or a combination of such networks. For example, wide area networks including the internet and the web can inter-communicate the information processing system 101 with other one or more information processing systems that may be locally, or remotely, located relative to the information processing system 101. It should be noted that mobile communications devices, such as mobile phones, Smart phones, tablet computers, lap top computers, and the like, which are capable of at least one of wired and/or wireless communication, are also examples of information processing systems within the scope of the present disclosure. The network interface device 116 can provide a communication interface for the information processing system 100 to access the at least one database 117 according to various embodiments of the disclosure.

The instructions 107, according to the present example, can include instructions for monitoring, instructions for analyzing, instructions for retrieving and sending information and related configuration parameters and data that would enable and facilitate an IoT powered e-charting system. It should be noted that any portion of the instructions 107 can be stored in a centralized information processing system or can be stored in a distributed information processing system, i.e., with portions of the system distributed and communicatively coupled together over one or more communication links or networks.

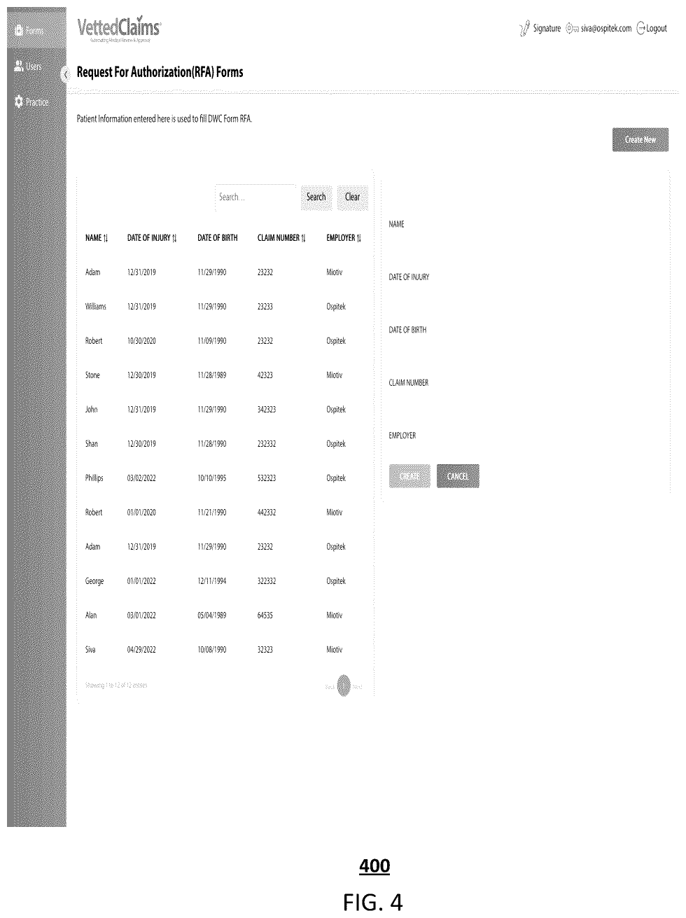

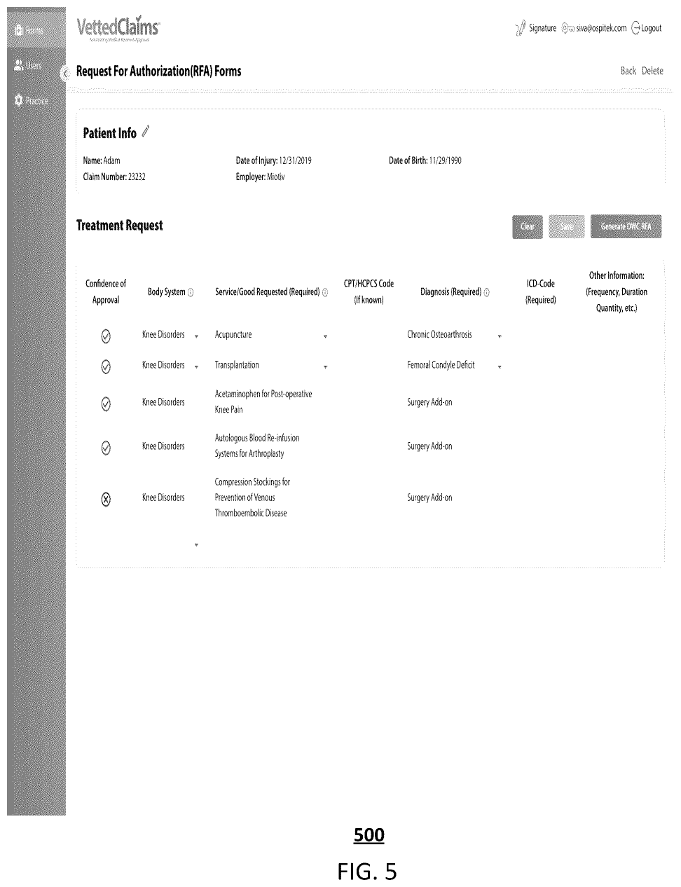

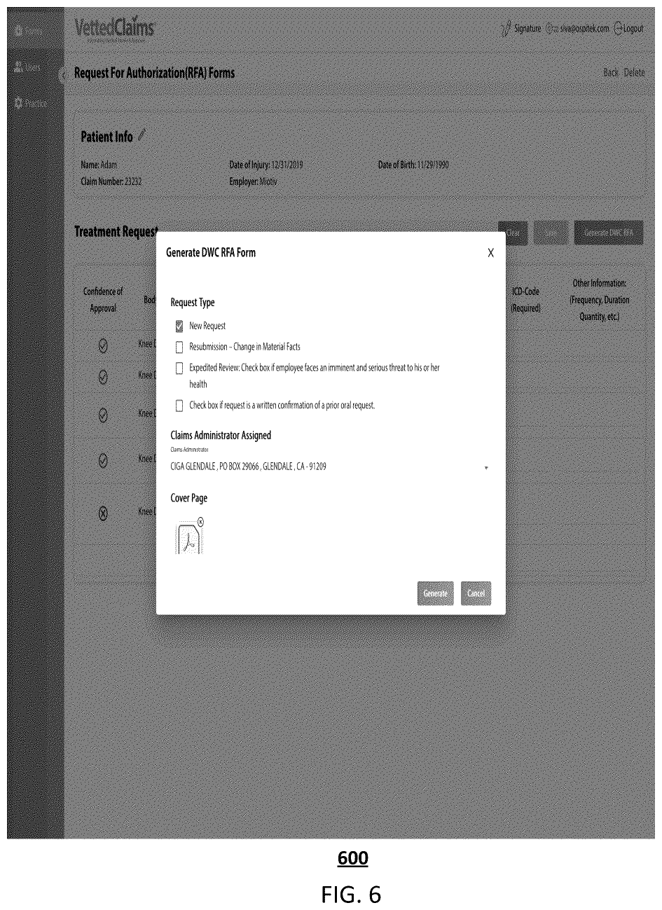

FIGS. 1-6 illustrate examples of systems (100), methods (600) or process flows, according to various embodiments of the present disclosure, which can operate in conjunction with the information processing system 700 or 101 of FIG. 7.

In some embodiments with reference to any of the embodiments, the various components can be arranged and configured to be in any number of parameters, positions and sizes as required for a particular embodiment. Some embodiments with smaller dimensions or parameters would likely be better suited for portable embodiments. For example, in a number of embodiments the forms 110 can be of any number of forms, not just the ones shown.

In interpreting the present disclosure and the claims, references of the form “A and/or B” encompass any and every combination and subcombination of the elements A and B, namely any or all of the following: (i.) A, (ii.) B, (iii.) A or B, and (iv.) A and B. References of the form “A, B, and/or C” likewise encompass any and every combination and subcombination of elements A, B, and C). Where the present disclosure or any of the claims may recite “a” or “a first” item or the equivalent thereof, such disclosure includes one or more such items and does not require or exclude two or more such items. Numerical or ordinal terms such as “first”, “second”, “third” etc. when used to refer to items are used solely to identify the items, and do not require or limit the number of such items elements and do not indicate, require or limit a particular position or order of such items unless expressly and clearly stated otherwise.

Descriptions made with reference to “embodiment”, “embodiments”, “some embodiments”, “an embodiment”, “preferred embodiment”, “other embodiments”, “alternative embodiments”, “various embodiments” or the like mean that the description is applicable to at least one embodiment but not necessarily all embodiments. The terms “comprising”, “including”, “having”, and the like, as used with respect to one or more embodiments, are synonymous. In some cases features, items steps or other subject matter are described herein as being optional or using terms such as “optional” or “optionally”. However, lack use of such terms in connection with the description of any other features, items steps or other subject matter does not in any way mean or imply that such other features, items steps or other subject matter are required or are not optional.

As an aid to understanding, various actions, operations or steps may sometimes be presented herein or described herein in sequence. However, the order of the description or written presentation herein is not to be construed to mean or imply that such must necessarily occur in a corresponding order or sequence unless otherwise expressly and clearly stated or logically essential. Some actions, operations or steps may permissibly be performed in an order or sequence other than the order of their description or written presentation herein unless otherwise expressly and clearly stated or logically essential. Unless otherwise expressly and clearly stated or logically essential. Unless otherwise expressly and clearly state or logically essential, actions, operations or steps described herein may be combined or divided. Unless otherwise expressly and clearly stated or logically essential, any description herein of any one or more actions, operations or steps does not preclude any one or more other preceding, succeeding and/or intervening actions, operations or steps irrespective of whether or not such preceding, succeeding and/or intervening actions, operations or steps are described or disclosed herein.

Unless otherwise expressly and clearly stated or logically essential, any illustration, description, or reference herein of any one or more items, structures or elements being “connected to”, “coupled to”, “joined to”, “joined with”, “attached to”, “mounted to”, “mounted in”, or “secured to” any one or more other specified items, structures or elements shall not be construed to preclude such connection, coupling, joint, attachment, mounting or securement being either made indirectly, by way of one or more other specified or unspecified items structures or elements, or being made directly.

Unless otherwise expressly and clearly stated or logically essential, any illustration, description, or reference herein of any one or more items, structures, or elements “adjoining”, any one or more other specified items, structures or elements, shall be construed to permit that such may adjoin either direct or indirectly. The term “adjoining” permits, but does not require, preclude the presence of items, structures or elements interposed between those describes as adjoining. Unless otherwise expressly and clearly stated or logically essential, any illustration, description, or reference herein to any one or more items, structures or elements being “beneath”, “below”, “above”, “behind”, “in front of”, “between”, “under”, “over”, “in”, “within”, “outside”, “inside”, any one or more other specified items, structures or elements and/or any other prepositions or prepositional phrases shall construed in a manner which permits, but does not require, contact or immediacy and any and all other prepositions and/or prepositional phrases shall be construed in that same manner.

While the embodiments have been described with reference to various preferred embodiments, it will be understood by those skilled in the art that various changes may be made and equivalents substituted for elements thereof without departing from the scope of the embodiments and that modifications may be made to adapt to a particular situation or application of the embodiments without departing from the scope. The embodiments within the scope of the claims are not limited to the particular embodiments disclosed. Rather, the claims cover all embodiments which are within the scope of the claims, either literally or under the Doctrine of Equivalents.

1. A system for vetting insurance authorization requests and improving chances of approval, comprising:
one or more processors;
memory coupled to the one or more processors, the memory containing computer instructions which when executed by the one or more processors, causes the one or more processors to:
retrieve and extract information from an authorization request including a treatment and a diagnosis;
identify treatment guidelines based on the information retrieved and extracted from the authorization request;
use rules that have been converted to a standard format that is human readable and separately maintain the rules from each authority issuing the rules;
apply rules to provide a confidence of approval value or indicator;
present the confidence value or indicator on a user interface based on the rules applied; and
present a submission button for generating a request for authorization form.

2. The system of claim 1, wherein the one or more processors are further configured to index the treatment and the diagnosis from the authorization request.

3. The system of claim 1, wherein the one or more processors are further configured to fetch a rule file or rule files based on the treatment in the authorization request.

4. The system of claim 1, wherein the one or more processors are further configured to determine if an approval is unconditional or conditional.

5. The system of claim 4, wherein the one or more processors are configured to indicate the approval if the approval is unconditional or all conditional requirements are met.

6. The system of claim 4, wherein the one or more processors are configured to solicit for more information to determine claim status if the approval is conditional.

7. The system of claim 1, wherein the one or more processors are further configured to use configuration files to determine which rule files apply.

8. The system of claim 1, wherein the one or more processors are configured to determine if the rule file is a latest version.

9. The system of claim 1, wherein the one or more processors are configured to present an excerpt of the rule file applied to allow determination of why a claim or authorization is approved or denied.

10. The system of claim 1, wherein presenting the confidence value or indicator on the user interface based on the rules applied comprises providing an estimated percentage of chance that a claim will be approved.

11. The system of claim 1, wherein the one or more processors are configured to track claims and feedback to provide an improved prediction analysis.

12. The system of claim 1, wherein the one or more processors are configured to iteratively determine and present the confidence value or indicator on the user interface based on the rules applied as the claim is presented and modified.

13. The system of claim 1, wherein the one or more processors are configured to provide a submission score for every authorization request submitted where the score is dependent on a probability that the authorization request adheres to the rules or insurance guidelines.

14. A system for vetting insurance authorization requests and improving chances of approval, comprising:
a server; and
one or more applications executing on the server that:
retrieves and extracts information from an authorization request including a treatment and a diagnosis;
identifies treatment guidelines based on the information retrieved and extracted from the authorization request;
uses rules that have been converted to a standard format that is human readable;
apply rules to provide a confidence of approval value or indicator; and
present the confidence value or indicator on a user interface based on the rules applied.

15. The system of claim 14, wherein the authorization request is created with information from a first questionnaire from a patient that includes primarily patient demographic information from the patient.

16. The system of claim 15, wherein the authorization request is further created with physician examination notes including CPT codes and IDC10 codes.

17. The system of claim 16, wherein the authorization request is further created with uploaded files and lab tests.

18. The system of claim 16, wherein a second questionnaire is generated and sent to the patient based on the CPT codes entered by the physician.

19. The system of claim 14, wherein the system provides a submission score for every authorization request submitted where the score is dependent on a probability that the authorization request adheres to the rules or insurance guidelines where a red indicator indicates a high probability of rejection of the authorization request and a green indicator indicates a high probability of allowance of the authorization request.

20. A method of vetting insurance authorization requests and improving chances of approval comprising an application at a server having one or more applications executing on the server which perform the functions of:
retrieving and extracting information from an authorization request including a treatment and a diagnosis;
identifying treatment guidelines based on the information retrieved and extracted from the authorization request;
applying rules that have been converted to a standard format that is human readable to provide a confidence of approval value or indicator; and
presenting the confidence value or indicator on a user interface based on the rules applied.
